(19) United States
(12) Patent Application Publication
(10) Pub. No.: US 2025/0143858 A1
(43) Pub. Date: May 8, 2025

(54) SYSTEMS AND METHODS FOR REINFORCEMENT OF BIOLOGICAL CHANNELS

(71) Applicant: Dignity Health, San Francisco, CA (US)

(72) Inventors: Tyler Cole, San Francisco, CA (US); Dakota Graham, San Francisco, CA (US)

(21) Appl. No.: 18/728,019

(22) PCT Filed: Jan. 23, 2023
(86) PCT No.: PCT/US23/61117

§ 371 Date: Jul. 10, 2024

Related U.S. Application Data
(60) Provisional application No. 63/301,778, filed on Jan. 21, 2022.

(51) Int. Cl. A61F 2/00; A61B 17/00; A61B 17/12; A61B 50/33

(52) U.S. Cl. CPC A61F 2/0063; A61B 17/12009; A61B 50/33; A61B 2017/00495; A61F 2210/0085

(57) ABSTRACT

Methods of externally reinforcing biological vessels include forming a reinforcing frame around a vessel using a pliable framing material, and applying a solidifying substance to the framing material that transitions the framing material and solidifying substance to a solid state, forming a solid encasement. The framing material can include a plurality of sheets for application in an overlapping piecemeal fashion to a vessel within a surgical corridor. The solidifying substance permeates the reinforcing frame prior to transitioning to the solid state to ensure that the resulting encasement is filly solidified.

### CROSS-REFERENCE TO RELATED APPLICATIONS

This is a PCT Patent Application that claims benefit to U.S. Provisional Patent Application Ser. No. 63/301,778 filed 21 Jan. 2022, which is herein incorporated by reference in its entirety.

### FIELD

The present disclosure generally relates to aneurysm treatment, and in particular, to a system and associated methods for reinforcement of biological channels.

### BACKGROUND

According to the Brain Aneurysm Foundation, ruptured brain aneurysms are fatal in about 50% of cases, and of those who survive, about 66% suffer some permanent neurological deficit. Current techniques of addressing aneurysm treatment are limited to open surgical clip occlusion, various endovascular modalities, and, least commonly, cotton-wrapping of the aneurysm for difficult-to-treat lesions. Other modalities of aneurysm encasement have been described, such as using methyl methacrylate cement, however, due to complications associated with re-treatment with this approach, these techniques have been abandoned (Ogilvy CS, Poletti CE. Clipping of an aneurysm 20 years after encasement with methyl methacrylate. Case report. J Neurosurg. 1991; 75:305-307). Further, some recent studies have determined that aneurysm recurrence occurs in about 20% of cases, with re-treatment being performed in about 10% of cases. As such, there exists a clinical need for better ways to reinforce complex aneurysms and prevent rupture, especially for cases where endovascular treatment is not favorable.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIGS. 1A-1C are a series of illustration showing various unique aneurysm topologies;

FIG. 2 is an illustration showing a kit for vessel reinforcement;

FIGS. 3A-3F are a series of illustrations showing various configurations of a framing material of the kit of FIG. 2;

FIGS. 4A-4G are a series of illustrations showing various material types of the framing material of the kit of FIG. 2;

FIGS. 5A-5C are a series of illustrations showing initial wrapping of the framing material of the kit of FIG. 2 around the various aneurysm topologies shown in FIGS. 1A-1C;

FIGS. 6A-6C are a series of illustrations showing a reinforcing frame constructed by wrapping the framing material around the various aneurysm topologies shown in FIGS. 5A-5C;

FIGS. 7A-7H are a series of illustrations showing various substance delivery mechanisms for delivering a solidifying substance of the kit of FIG. 2 to the framing material;

FIGS. 8A-8D are a series of illustrations showing a first sequence for vessel reinforcement by the kit of FIG. 2;

FIGS. 9A-9D are a series of illustrations showing a second sequence for vessel reinforcement by the kit of FIG. 2;

FIGS. 10A-10D are a series of illustrations showing a third sequence for vessel reinforcement by the kit of FIG. 2;

FIGS. 11A-11D are a series of illustrations showing a fourth sequence for vessel reinforcement by the kit of FIG. 2;

FIGS. 12A-12D are a series of illustrations showing a fifth sequence for vessel reinforcement by the kit of FIG. 2;

FIGS. 13A-13D are a series of illustrations showing testing of vessel reinforcement with a 3-D printed vessel following the sequence of FIGS. 8A-8D;

FIGS. 14A-14F are a series of illustrations showing testing of vessel reinforcement with a cadaver vessel following the sequence of FIGS. 8A-8D;

FIGS. 15A-15F are a series of illustrations showing testing of vessel reinforcement with a 3-D printed vessel following the sequence of FIGS. 9A-9D; and

FIG. 16 is a process flow diagram showing a method for providing a kit for vessel reinforcement according to FIGS. 2-15F.

Corresponding reference characters indicate corresponding elements among the view of the drawings. The headings used in the figures do not limit the scope of the claims.

### DETAILED DESCRIPTION

A system provides materials and techniques for external aneurysm reinforcement that avoid the need for bypass revascularization, arrest the growth of the aneurysm, and serve as a barrier to prevent rupture or reduce internal bleeding in case of rupture.

The system can include one or more sheets of a framing material that are of a biocompatible and conformable material for circumferential placement around a vessel. The framing material can be wrapped around and/or applied in an overlapping piecemeal fashion to the vessel to form a reinforcing frame around the vessel. Further, the system can include a solidifying substance that, when applied to and absorbed by the reinforcing frame, solidifies the reinforcing frame around the vessel, thereby forming an encasement. In some embodiments, the framing material can be embedded with a reactant substance that, when combined with a catalyst of the solidifying substance, causes the reinforcing frame to solidify around the vessel and form an encasement.

In some embodiments, the present system may be offered as a kit to enable practitioners to reinforce vessels on a case-by-case basis, such that a practitioner may position the one or more sheets around a vessel having a unique morphology. The one or more sheets of the framing material may be pre-cut in various shapes and sizes for effective piecemeal positioning around a vessel to form the reinforcing frame.

Further, the solidifying substance can be applied to the framing material prior to or following positioning of the framing material around the vessel, depending on whichever sequence the practitioner may deem appropriate.

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

References to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments. Thus, the present technology can include a variety of combinations and/or integrations of the embodiments and features described herein.

As used herein, direction or relational terms such as “along,” “around,” and “circumferentially,” are used to facilitate understanding and discussion, not limitation. For example, these terms generally provide context for viewing embodiments and understanding relative positions and orientations of associated components.

Further, quantifying terms such as “sufficient”, “substantial” and “highly” may be used herein to generalize the disclosed relationships and relative quantities of components and substances to account for minor design modifications/alterations that do not depart from the spirit and scope of this disclosure. As used herein, the term “sufficiently” can be interpreted as exhibiting enough of a quantity or quality as understood by one skilled in the art. For example, two substances being “sufficiently combined” can be interpreted as combined to a degree that one skilled in the art would understand as being sufficient. In another example, a substance being “sufficiently permeated” with respect to a material can be interpreted as having saturated through the material to a degree that one skilled in the art would understand as being sufficient. A component being “sufficiently overlapping” with respect to another component can similarly be interpreted as overlapping the other component to a degree that one skilled in the art would understand as being sufficient. Further, a “sufficient quantity” of a material or substance can be interpreted as having enough of the material or substance to a degree that one skilled in the art would understand as being sufficient.

In yet another example, a substance or material being “highly permeable”, “highly absorbable”, “highly biocompatible”, or variations thereon, can be respectively interpreted as permeable, absorbable, or biocompatible to a degree that one skilled in the art would understand as exhibiting a high or otherwise significant degree of permeability, absorbability, or biocompatibility.

Finally, it is also appreciated that the illustrated devices and structures may include a plurality of the same component referenced by the same number. It is appreciated that depending on the context, the description may interchangeably refer to an individual component or use a plural form of the given component(s) with the corresponding reference number.

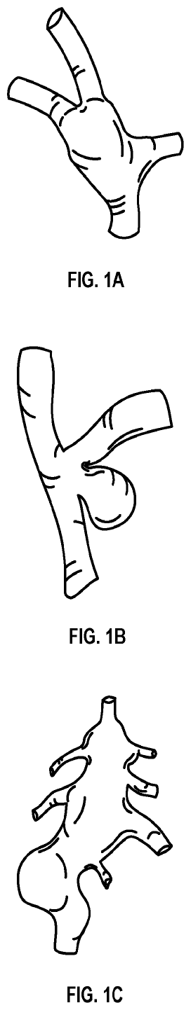

FIGS. 1A-1C show various examples of diseased vessels having complex aneurysm shapes that may be treated using the systems and methods outlined herein. As shown, complex aneurysms can include bulging areas along a vessel where one or more “trunks” or “branches” meet. These types of aneurysms are not easily treated using conventional methods such as clipping, flow diversion or embolization. Complications of conventional treatment methods can include flow occlusion, clotting, re-occurrence or expansion of the aneurysm.

Previous works have explored the use of methyl methacrylate cement applied directly to biological vessels to form encasements; however, these methods have been long abandoned due to problems with re-treatment. Other methods that have been explored include wrapping biological vessels in cotton or a similar material and placing a clip around the vessel; these methods can still have adverse effects, and are not always effective for more complex aneurysm topologies. Endovascular treatment is not always favorable as this method requires blood thinning medications that increase the risk of hemorrhage. This is because endovascular devices left within the blood vessel can form clots, leading to stroke. Alternatively, open vascular procedures such as extracranial to intracranial bypasses avoid blood thinning medications, but the overall complication risk of these procedures is particularly high. As such, an object of the present disclosure is to provide methods and tools for external reinforcement of biological vessels that avoids the need for endovascular device placement, avoids risk associated with bypass revascularization, arrests the growth of the aneurysm, and serves as a barrier to prevent rupture or the spread of blood in case of rupture.

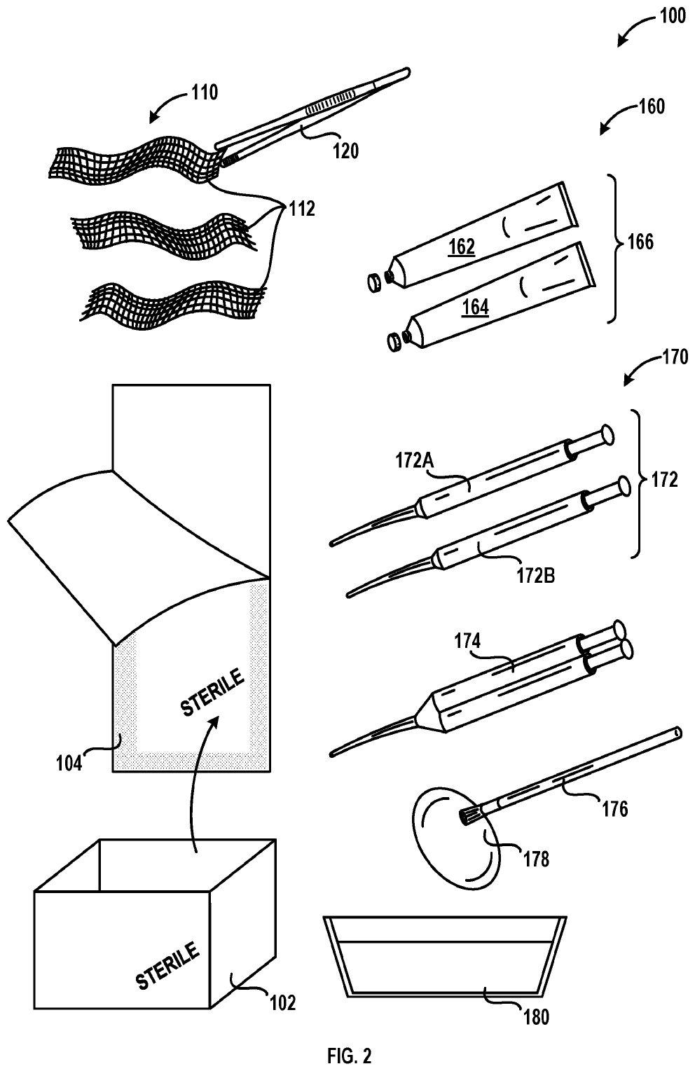

FIG. 2 shows a system for external aneurysm reinforcement that may be embodied as a kit, e.g., kit 100, including a framing material 110 that may be provided as a plurality of sheets 112, and a solidifying substance 160 that may include one or more applicator tools 170. The framing material 110 can be wrapped around and/or applied in an overlapping piecemeal fashion to the vessel to form a reinforcing frame around the vessel. The solidifying substance 160 can be applied to the framing material 110 before, during, and/or after placement of the framing material 110 along the vessel to fully permeate the framing material 110 and transition the framing material 110 and solidifying substance 160 to a solid state, thereby forming an encasement.

A kit embodiment of the present concept may be particularly advantageous for deploying the inventive concept as a portable (preferably disposable) package in a surgical environment or other such location. In general, the kit 100 is suitable for external aneurysm reinforcement, and includes by non-limiting examples, a container 102, such as a box defining a housing or cavity and cover for secure storage and transportation of the framing material 110 and the solidifying substance 160. In some embodiments, container 102 can be sterile. Further, the framing material 110, the solidifying substance 160, and any associated tools and/or applicators can be individually or collectively packaged within one or more packaging units 104 that may be positioned within the container 102 to ensure sterility, lack of tampering, and safe transport.

The framing material 110 can be of a flexible, conformable, biocompatible and porous material such as woven or non-woven fabric. It is desired that the framing material 110 can loosely conform around the vessel so as not to constrict blood flow. As shown, the kit 100 can include one or more framing tool(s) 120 such as tweezers, forceps, or another suitable tool that enable a practitioner to grasp and manipulate the framing material 110 during placement around a vessel. Framing tool(s) 120 may be elongated and may be shaped for optimal delivery of the framing material 110 within the surgical corridor. In a further aspect, the kit 100 can include a cutting tool that enables a practitioner to cut the framing material 110 into custom shapes and sizes that are ideal for the specific morphology of the vessel. The framing material 110 can be configured for positioning around the vessel to form a reinforcing frame around the vessel, e.g., by wrapping the framing material 110 around the vessel and/or applying the framing material 110 to the vessel in an overlapping piecemeal fashion.

The solidifying substance 160 can include one or more liquid materials that transition to a solid material; the solidifying substance 160 can be a single-part liquid that transitions to a solid material, or can be a multi-part substance including at least one liquid that, when combined, transitions to a solid material. As shown, the solidifying substance 160 can include a first substance 162, and optionally a second substance 164, both of which may be respectively stored in separate tubes 166 as shown.

The kit 100 can include one or more applicator tool(s) 170 for application of the solidifying substance 160 to the framing material 110. The one or more applicator tool(s) 170 can include one or more single-barrel syringes 172 for storage, transport and/or application of one or more individual components of the solidifying substance 160 (e.g., a first single-barrel syringe 172A for application of first substance 162 and a second single-barrel syringe 172B for application of second substance 164). The one or more applicator tool(s) 170 can also include one or more double-barrel syringes 174 for storage, transport, mixing and/or simultaneous application of two or more individual components of the solidifying substance 160 (e.g., first substance 162 and second substance 164). Other components of the kit 100 can include an applicator brush 176 and an associated applicator palette 178 for mixing and/or application of the solidifying substance 160. In a further aspect, the kit 100 can include a soaking tray 180 for soaking the framing material 110 within one or more components of the solidifying substance 160 prior to application of the framing material 110 around the vessel.

The kit 100 provides various options for practitioners to reinforce complex aneurysms as the practitioner sees fit. In one example, a practitioner may prefer to apply one or more components of the solidifying substance 160 to the framing material 110 prior to wrapping the framing material 110 around the vessel. In another example, a practitioner may prefer to wrap the framing material 110 around the vessel prior to application of the solidifying substance 160 to the framing material 110. The framing material 110 and the solidifying substance 160 can be selected by the practitioner for optimal pairing based on complimentary characteristics of each. For instance, pairing the solidifying substance with the correct framing material is important to ensure absorption, especially taking into account hydrophilic/hydrophobic properties of the materials. For example, a solidifying substance that includes a PPODA-QT mixture pairs well with a framing material that includes polypropylene fabric because polypropylene fabric thoroughly absorbs PPODA-QT since both materials are hydrophobic.

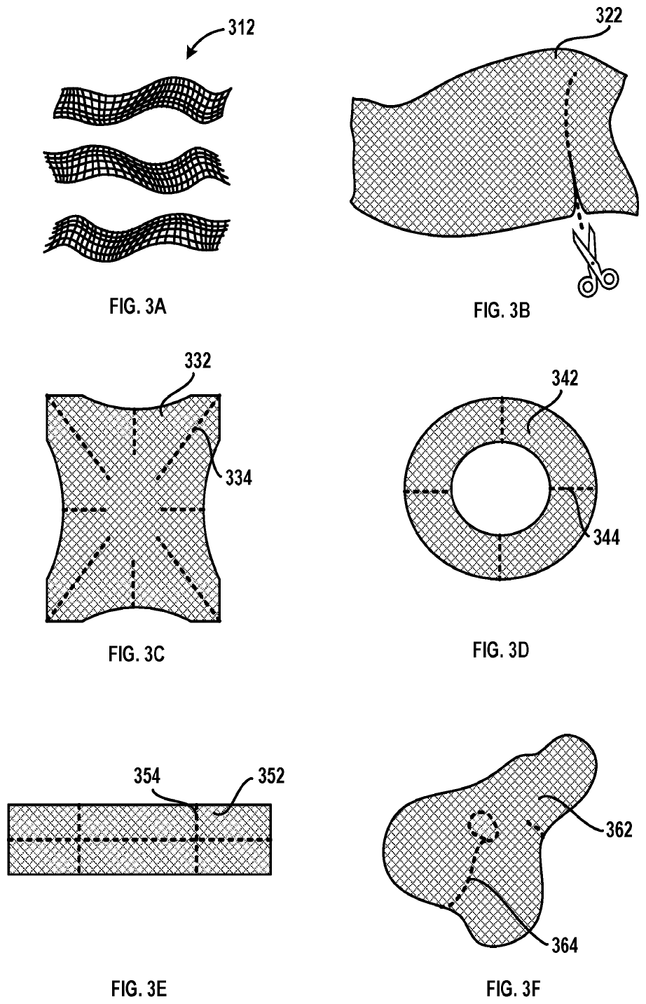

FIGS. 3A-3F show various configurations and shapes of framing material (e.g., framing material 110) that can be provided within a kit (e.g., kit 100) for aneurysm reinforcement. As shown in FIG. 3A, the framing material can be provided as a plurality of sheets 312 for piecewise application to a vessel. FIG. 3B shows a single sheet 322 of framing material that a practitioner can cut to size as they see for. FIGS. 3C-3F show various pre-cut and/or perforated sheets. FIG. 3C in particular shows a first perforated sheet 332 of framing material having angular perforations 334 that aid in conforming the framing material to a vessel; angular perforations 334 can also act as cut lines. FIG. 3D shows a second perforated sheet 342 of framing material having a generally circular configuration and having perforations 344 that aid in conforming the framing material to a vessel. FIG. 3E shows a third perforated sheet 352 of framing material having a generally planar configuration and having perforations 354 that aid in conforming the framing material to a vessel. FIG. 3F shows a fourth perforated sheet 362 of framing material having an amorphous configuration and having perforations 364 that aid in conforming the framing material to a vessel. A practitioner can cut the framing material to size as needed in order to wrap or otherwise position the framing material in an overlapping, piecewise manner around unique vessel topologies. In some non-limiting examples, individual sheets of framing material can be dimensioned within a millimetric scale; for example, individual sheets can be about 16 mm long and 4 mm wide. However, due to variations in vessel morphology, appropriate sizes and shapes of individual sheets of framing material may vary.

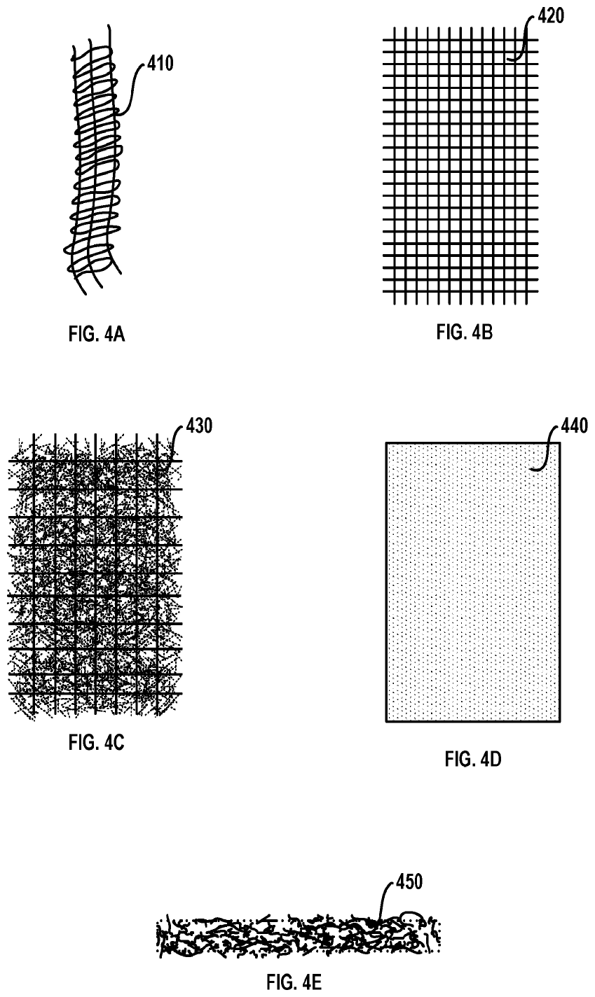

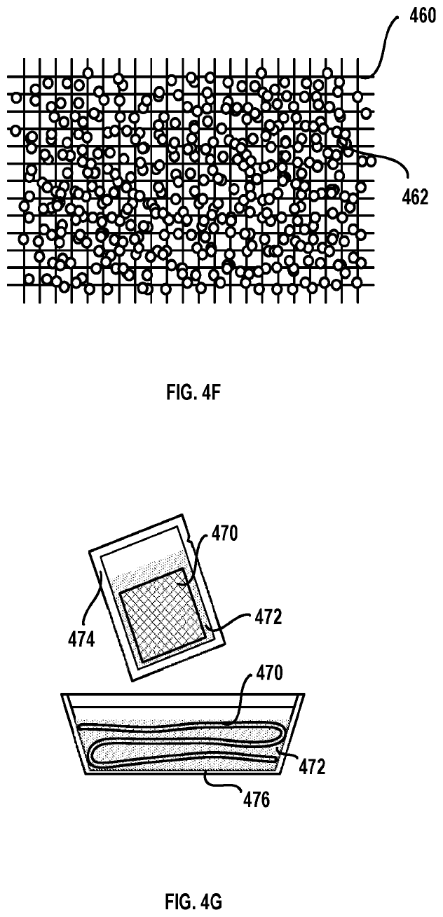

FIGS. 4A-4G show various fabric configurations of framing material (e.g., framing material 110) that can be provided within a kit (e.g., kit 100) for aneurysm reinforcement. The framing material can include a fabric and/or fiber material that, when positioned along a vessel in an overlapping piecewise arrangement, forms a reinforcing frame around the vessel. The framing material absorbs a solidifying substance (e.g., solidifying substance 160) that forms an encasement around the vessel after transitioning to a solid state. The framing material should be biocompatible and highly permeable and absorbent with respect to the solidifying substance. Further, the framing material should be flexible and conformable such that the framing material adheres loosely to the unique morphology of the vessel when applied. In one aspect, the framing material can adhere to itself. When wrapping the framing material around the vessel, it is imperative to ensure that the framing material sufficiently overlaps and “holds” itself together. The framing material can include woven or non-woven fabrics, and can be formed from materials including but not limited to polypropylene, cotton, wool and cellulose. The framing material should be biocompatible, and may be selected to provoke minimal or no local vessel inflammation. The framing material may be hydrophobic or hydrophilic; selection of the framing material can be guided with respect to a corresponding solidifying substance (e.g., if the framing material is hydrophobic, the solidifying substance should also be hydrophobic, highly absorbable by the framing material and able to fully permeate the framing material). Further, the structure of the framing material can be selected based on preferences of the practitioner.

FIG. 4A shows one example framing material 410 having a loose netting configuration. FIG. 4B shows another example framing material 420 having a woven fabric configuration. FIG. 4C shows an example framing material 430 having a spun-laid non-woven fabric configuration. FIG. 4D shows another example framing material 440 having a wet-laid non-woven fabric configuration. FIG. 4E shows a framing material 450 having a texturized surface where the fibers have been intentionally loosened; the framing material 450 can be pre-texturized during manufacture to make the framing material 450 more receptive to the solidifying substance.

FIG. 4F shows a framing material 460 having a first substance 462 embedded therein. The first substance 462 can be a liquid or dry material, and can be part of the solidifying substance such that, when combined with a second substance of the solidifying substance, the framing material 460 and solidifying substance transition into a solid state to form the encasement around the vessel. FIG. 4G shows a framing material 470 having been pre-soaked in a first substance 472; the first substance 472 can be a liquid, and can be part of the solidifying substance such that, when combined with a second substance of the solidifying substance, the framing material 470 and solidifying substance transition into a solid state to form the encasement around the vessel. In the embodiments shown, the framing material 470 can be provided within the kit in a tear-open pouch 474 or a single-use soaking tray 476 that can, for example, have a peel-off lid. First substances 462 and/or 472 can begin a reaction that causes contiguous portions of the associated framing material 460 and/or 470 to harden when combined with a second substance of the solidifying substance.

In some embodiments, the first substance may not necessarily be reactive with the second substance, and can serve to “prime” the framing material to be receptive to the second substance of the solidifying substance. For instance, the first substance can permeate the framing material and make the framing material more receptive to additional liquid materials such that the second substance can more easily permeate the framing material, ensuring that the solidifying substance is evenly dispersed throughout the resulting encasement and saturates the framing material.

In a further aspect, the framing material can be impregnated with a substance such as a medication or other chemicals to promote good surgical outcomes, such as anti-inflammatory or anti-bacterial substances. Further, in some examples, framing material can be impregnated with a material or drug that facilitates growth of scar tissue along an exterior of the vessel to form a bio-scaffold along the vessel.

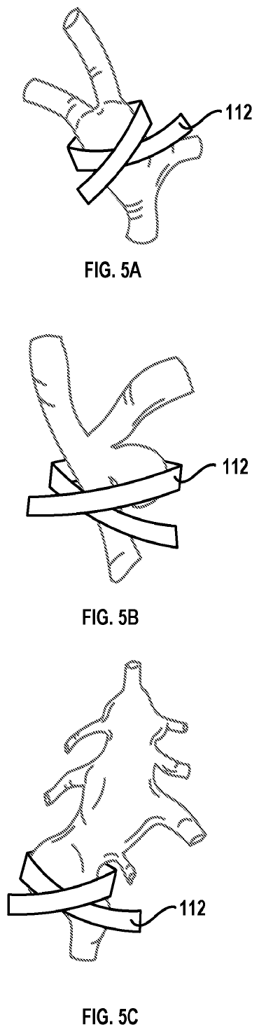

FIGS. 5A-5C show initial wrapping of individual sheets 112 of a framing material (e.g., framing material 110) around three diseased vessels having unique topologies to form a reinforcing frame around the vessel. As shown, when placing individual sheets 112 around the vessel, it is important that respective ends of each sheet 112 of the framing material contact one another in an overlapping fashion to ensure that the sheet 112 stays in place. As discussed, each sheet 112 can adhere to itself. In some embodiments, adherence of each respective sheet 112 to one another may be aided or otherwise facilitated by the solidifying substance (e.g., solidifying substance 160) when the solidifying substance is combined with the framing material. For example, a sheet 112 that is soaked in a liquid form of at least one substance of the solidifying substance may adhere to itself and/or other sheets through surface tension of the substance and/or through adhesive properties of the substance. During initial application of the framing material, each sheet 112 should be positioned loosely around the vessel to avoid compression of the vessel. By positioning individual sheets 112 around the vessel as opposed to one continuous sheet, the reinforcing frame can be more easily constructed within the surgical corridor. As discussed herein, the individual sheets 112 may be soaked in at least one part of the solidifying substance prior to application around the vessel; alternatively, the individual sheets 112 may be positioned around the vessel prior to application of at least one part of the solidifying substance, depending on the specific needs and/or preferences of the patient and/or practitioner.

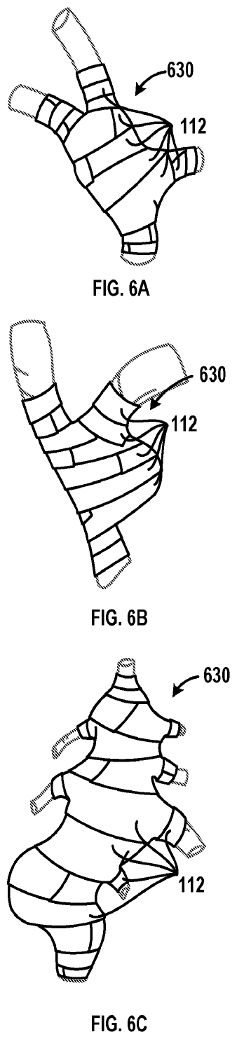

FIGS. 6A-6C show the framing material wrapped around the diseased vessels of FIGS. 5A-5C to form a reinforcing frame 630 around each diseased vessel. As shown, the reinforcing frame 630 is constructed by placing the plurality of sheets 112 sequentially and circumferentially around the vessel in a piecemeal fashion with overlapping edges. The reinforcing frame 630 can cover diseased portions of the vessel, and can extend to non-diseased parts of the vessel including connected paths to ensure full coverage. Depending on the sequence taken by the practitioner, the individual sheets 112 that form the reinforcing frame 630 may have been pre-soaked in at least one part of the solidifying substance prior to application; in other examples, the solidifying substance may be applied to the reinforcing frame 630 as will be discussed herein.

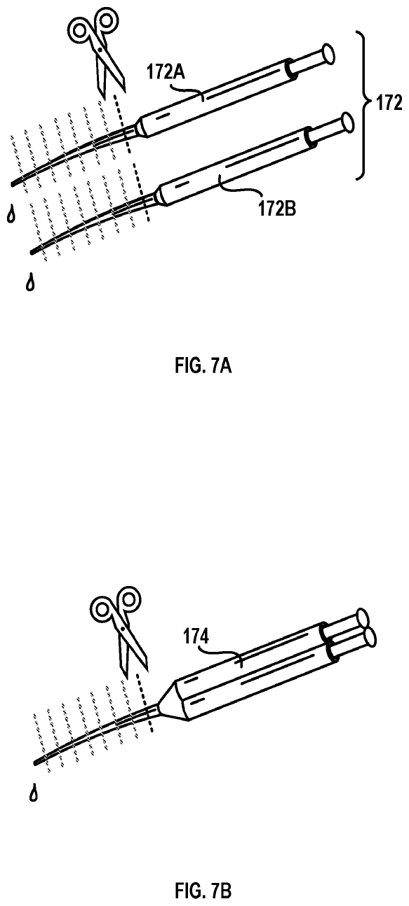

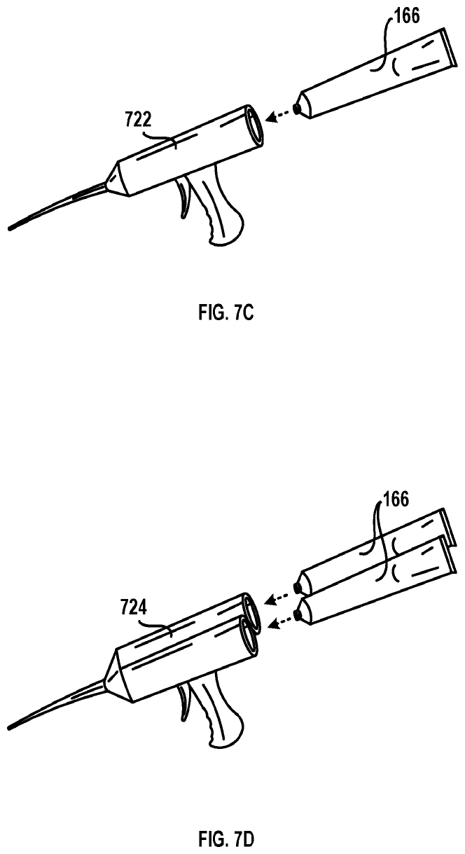

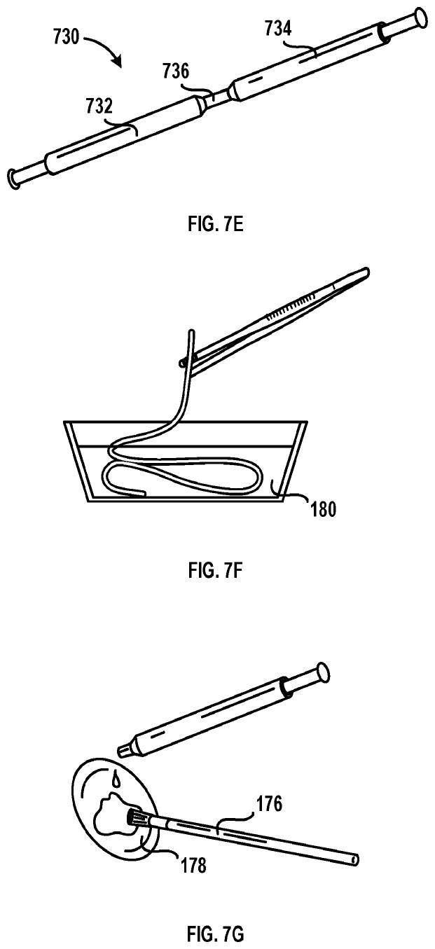

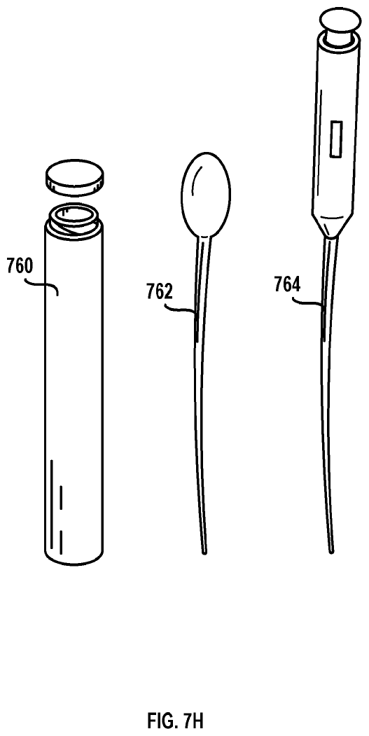

FIGS. 7A-7G show various ways that the solidifying substance (e.g., solidifying substance 160) can be mixed and/or applied to the framing material (e.g., framing material 110). In some embodiments, the solidifying substance can be a multi-part substance including a first substance (e.g., which may include first substance 162, 462 and/or 472 discussed above) and a second substance that, when combined and applied to the framing material, causes the solidifying substance and framing material to transition to a solid state, thereby forming an encasement around the vessel. The first substance and the second substance can be mixed as needed prior to application to the framing material; in other examples, the first substance and the second substance can be applied separately to the framing material. In some embodiments, the first substance of the solidifying substance can be pre-embedded within the framing material or can otherwise be packaged with the framing material such that the framing material is pre-soaked in the first substance. In other embodiments, the solidifying substance can be a single-part substance that hardens when applied to the framing material and following exposure to an environmental factor such as air or light. The solidifying substance should have a total curing time of 20 minutes or less to ensure that the resulting encasement is completely solidified by the end of a procedure.

The solidifying substance fully permeates the framing material upon application to ensure that the resulting encasement is fully solid. As such, the solidifying substance exhibits high absorption with respect to the framing material.

In a further aspect, the resulting encasement should be strong enough to withstand high blood pressure (˜180 mmHg) to prevent rupture. In some examples, the resulting encasement can be elastic enough to withstand high blood pressure and to avoid sharp edges when solidified, but should still be rigid enough to provide the reinforcing structure around the diseased vessel and prevent expansion of the aneurysm.

The solidifying substance can be of a single substance (e.g., not requiring combination of two or more substances), or can include at least a first substance and a second substance that must be combined. The solidifying substance can be applied to the framing material by one or more applicator tool(s) (e.g., applicator tool(s) 170) provided within a kit (e.g., kit 100) for vessel reinforcement.

In the example of FIG. 7A, the first substance and the second substance can be respectively provided within a first single-barrel syringe 172A and a second single-barrel syringe 172B as shown. The first single-barrel syringe 172A and the second single-barrel syringe 172B can each include an elongated applicator tip that can be cut to a preferred length if needed. As shown, the elongated applicator tips can be curved to aid in delivery of the first substance and/or the second substance to the framing material within the surgical corridor. In the example of FIG. 7B, the first substance and the second substance can be respectively provided within respective first and second barrels of a double-barrel syringe 174 as shown. The double-barrel syringe 174 can include an elongated applicator tip that can be cut to a preferred length if needed; when operating the double-barrel syringe 174, the first substance and the second substance can mix within the elongated applicator tip immediately prior to delivery. As shown, the elongated applicator tip can be curved to aid in delivery of the first substance and the second substance to the framing material within the surgical corridor.

In the examples of FIGS. 7C and 7D, the solidifying substance can be provided within the one or more tubes 166 (either as a single-part substance within a single tube 166 or a multi-part substance within multiple separate tubes 166) for delivery by one or more applicator tool(s) 170. In FIG. 7C, the one or more applicator tool(s) 170 can include a single-barrel trigger-aided applicator tool 722 that receives an associated tube 166 for delivery of one or more parts of the solidifying substance to the framing material within the surgical corridor. In FIG. 7D, the one or more applicator tool(s) 170 can include a double-barrel trigger-aided applicator tool 724 that receives two or more associated tubes 166 for delivery of one or more parts of the solidifying substance to the framing material within the surgical corridor. In some embodiments, the one or more tubes 166 include a foil tip that can be punctured upon insertion of the one or more tubes 166 into the associated single-barrel trigger-aided applicator tool 722 or double-barrel trigger-aided applicator tool 724. The single-barrel trigger-aided applicator tool 722 and/or double-barrel trigger-aided applicator tool 724 can include an elongated applicator tip that can be cut to a preferred length if needed; when operating the double-barrel trigger-aided applicator tool 724, the first substance and the second substance can mix within the elongated applicator tip immediately prior to delivery. The single-barrel trigger-aided applicator tool 722 and/or double-barrel trigger-aided applicator tool 724 can be electronically aided to ensure controlled delivery of the solidifying substance.

FIG. 7E shows a syringe-based mixing tool 730 that may be provided within the kit for mixing constituent parts of the solidifying substance. The syringe-based mixing tool 730 can include a first chamber 732 and a second chamber 734 removably coupled at a linkage 736; the first chamber 732 can be partially filled with the first substance and the second chamber 734 can be partially filled with the second substance. The first chamber 732 and second chamber 734 can communicate with one another when coupled at the linkage 736 such that “pushing” the first substance out of the first chamber 732 causes the first substance to enter the second chamber 734 and mix with the second substance. Likewise, “pushing” the second substance out of the second chamber 734 causes the contents of the second chamber 734 to enter the first chamber 732. Repeating this process several times, can allow sufficient mixing of the first substance and the second substance prior to delivery. In some embodiments, when the first substance and the second substance are sufficiently mixed, the mixture may be collected within the first chamber 732 or the second chamber 734 and the linkage may be replaced with an elongated applicator tip. In other embodiments, the mixture may be released into another container for application to the framing material by the one or more applicator tool(s).

FIG. 7F shows the soaking tray 180 of the one or more applicator tool(s) that may be included within the kit for applying one or more components of the solidifying substance to the framing material prior to application of the framing material to the vessel. In embodiments where the solidifying substance includes a single substance, a sufficient quantity of the solidifying substance can be placed into the soaking tray 180 along with the framing material to saturate the framing material with the solidifying substance prior to application of the framing material to the vessel. In embodiments where the solidifying substance includes a first substance and a second substance, but the first substance is embedded within the framing material as in some examples outlined above, then the second substance can be placed into the soaking tray 180 along with the framing material to saturate the framing material with the second substance and to combine the first substance with the second substance at the framing material prior to application of the framing material to the vessel. In embodiments where the solidifying substance includes a first substance and a second substance, but the practitioner wants to saturate the framing material with only the first substance prior to application of the framing material to the vessel, then the first substance can be placed into the soaking tray 180 along with the framing material to saturate the framing material with the first substance prior to application of the framing material to the vessel; following application of the framing material and the first substance to the vessel, the second substance can be applied to the framing material. In embodiments where the solidifying substance includes a first substance and a second substance, and the practitioner wants to saturate the framing material with the fully-combined first substance and second substance prior to application of the framing material to the vessel, then the first substance and the second substance can be mixed and placed into the soaking tray 180 along with the framing material to saturate the framing material with the first substance and the second substance prior to application of the framing material to the vessel. Finally, in some embodiments, the soaking tray 180 may be one of a plurality of soaking trays for individual receipt of the first substance and the second substance of the solidifying substance such that the framing material can be soaked within the first substance, and then subsequently dipped into the second substance prior to application of the framing material to the vessel.

FIG. 7G shows the applicator brush 176 and palette 178 of the one or more applicator tool(s) that may be employed to mix constituent parts of the solidifying substance and/or apply one or more parts of the solidifying substance to the framing material. Even if other applicator tools are primarily used to mix and apply the solidifying substance to the framing material, the applicator brush 176 can be used as needed within the surgical space to ensure complete and/or even coverage and saturation of the framing material with the solidifying substance. The applicator brush 176 can also be used with caution to clean up any residual or errant placement of the solidifying substance within the space. As such, the applicator brush 176 may have very soft bristles to avoid tissue abrasion and to hold more of the solidifying substance when being used to apply the solidifying substance within the surgical space.

FIG. 7H shows a bottle 760 and pipettes of the one or more applicator tool(s) that may be provided within the kit for measuring, mixing and/or delivering the solidifying substance. The pipettes can include a manual pipette 762 and/or a precision pipette 764 that may be electronically controlled or otherwise capable of measuring and/or delivering one or more parts of the solidifying substance in precise quantities. The bottle 760 can be one of a plurality of bottles that may include respective first and second substances of the solidifying substance for combination by the practitioner immediately prior to application to the framing material. The pipettes 762 and/or 764 can be employed to carefully measure quantities of the solidifying substance (including respective quantities of the first substance and the second substance) prior to application and/or combination to ensure a proper ratio. Optionally, the pipettes 762 and/or 764 can be employed to deliver the solidifying substance to the framing material.

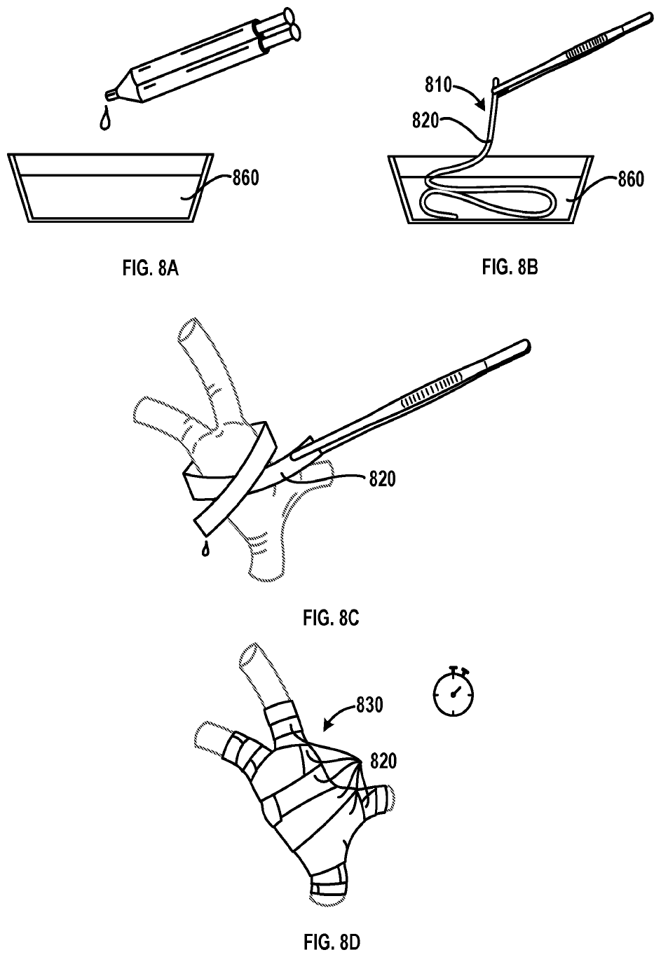

FIGS. 8A-8D show a first sequence for vessel reinforcement using the kit outlined herein. As shown in FIGS. 8A and 8B, a solidifying substance 860 can be combined and subsequently applied to a sheet 820 of a plurality of sheets 820 of a framing material 810 as shown. The solidifying substance 860 can include a first substance and a second substance that are combined to form the solidifying substance 860; upon combination, the first substance and the second substance can start a reaction that will eventually transform the solidifying substance 860 from a liquid form to a solid form. In some examples, the solidifying substance 860 can instead be of single substance (e.g., not requiring combination of two or more substances). The sheets 820 can be saturated within the solidifying substance 860 while the solidifying substance 860 is still in liquid form.

Then, as shown in FIG. 8C, the sheets 820 can be applied in-situ in an overlapping, piecemeal arrangement around the vessel to form the reinforcing frame 830. Once the reinforcing frame 830 is complete, as shown in FIG. 8D, then the reinforcing frame 830 can be allowed to cure until the resulting encasement is fully solidified around the vessel. Soaking each sheet 820 in the fully-combined solidifying substance 860 ensures that the entire reinforcing frame 830 is fully saturated in the solidifying substance 860.

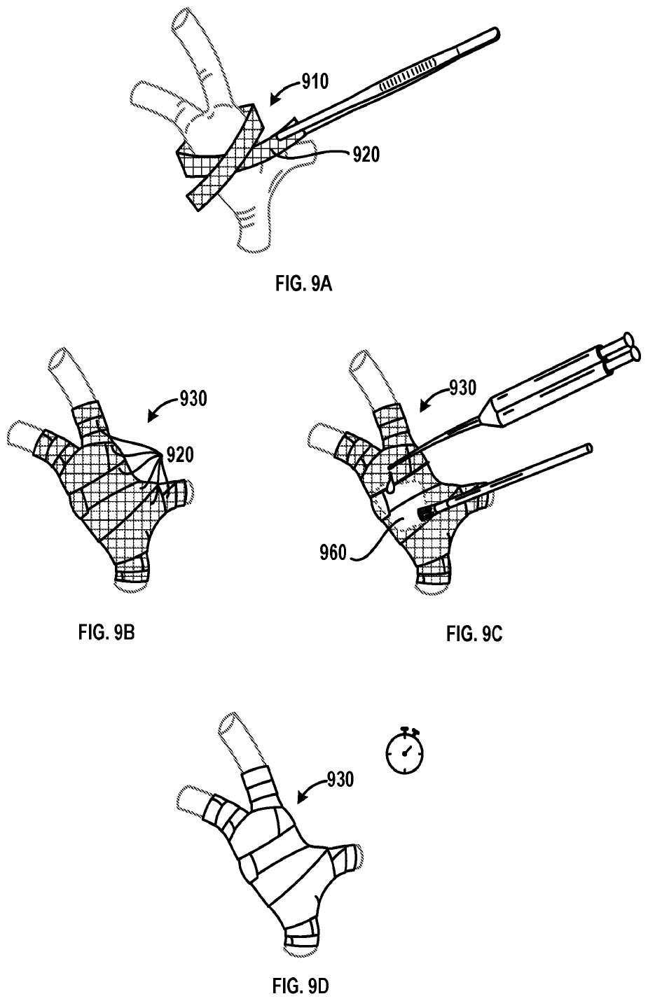

FIGS. 9A-9D show a second sequence for vessel reinforcement using the kit outlined herein. As shown in FIGS. 9A and 9B, a plurality of sheets 920 of a framing material 910 can be applied in-situ in an overlapping, piecemeal arrangement around a vessel to form a reinforcing frame 930. Following formation of the reinforcing frame 930, a solidifying substance 960 can be applied to the reinforcing frame 930 until the reinforcing frame 930 is fully saturated. The solidifying substance 960 can include a first substance and a second substance that are combined to form the solidifying substance 960; upon combination, the first substance and the second substance can start a reaction that will eventually transform the solidifying substance 960 from a liquid form to a solid form. In some examples, the solidifying substance 960 can instead be of single substance (e.g., not requiring combination of two or more substances). Care should be taken to ensure that the entirety of the reinforcing frame 930 is fully saturated with the solidifying substance 960. Once the reinforcing frame 930 is complete and has been fully saturated with the solidifying substance 960, as shown in FIG. 9D, then the reinforcing frame 930 can be allowed to cure until the resulting encasement is fully solidified around the vessel.

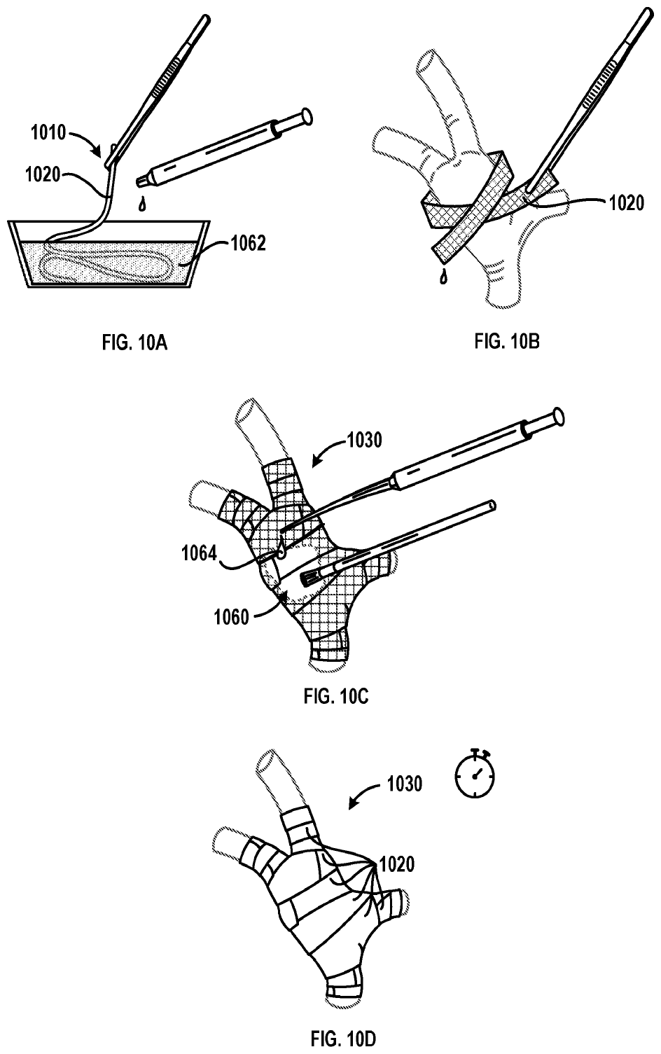

FIGS. 10A-10D show a third sequence for vessel reinforcement using the kit outlined herein. As shown in FIG. 10A, a plurality of sheets 1020 of a framing material 1010 can first be soaked in a first substance 1062 of a solidifying substance 1060 until fully saturated. In some examples, the plurality of sheets 1020 can already be pre-soaked within the first substance 1062. Then as shown in FIG. 10B, the sheets 1020 can then be applied in-situ in an overlapping, piecemeal arrangement around a vessel to form a reinforcing frame 1030. Following formation of the reinforcing frame 1030, as shown in FIG. 10C, a second substance 1064 of the solidifying substance 1060 can be applied to the reinforcing frame 1030 having the first substance 1062 until the reinforcing frame 1030 is fully saturated with the combined first substance 1062 and the second substance 1064. Once the reinforcing frame 1030 is complete and has been fully saturated with the solidifying substance 1060, as shown in FIG. 10D, then the reinforcing frame 1030 can be allowed to cure until the resulting encasement is fully solidified around the vessel.

In some embodiments, the first substance 1062 may not necessarily be reactive with the second substance 1064, and can serve to “prime” the framing material 1010 to be receptive to the second substance 1064 of the solidifying substance 1060. For instance, the first substance 1062 can permeate the framing material 1010 and make the framing material 1010 more receptive to additional liquid materials such that the second substance 1064 can more easily permeate the framing material 1010, ensuring that the solidifying substance 1060 is evenly dispersed throughout the resulting encasement and saturates the framing material 1010.

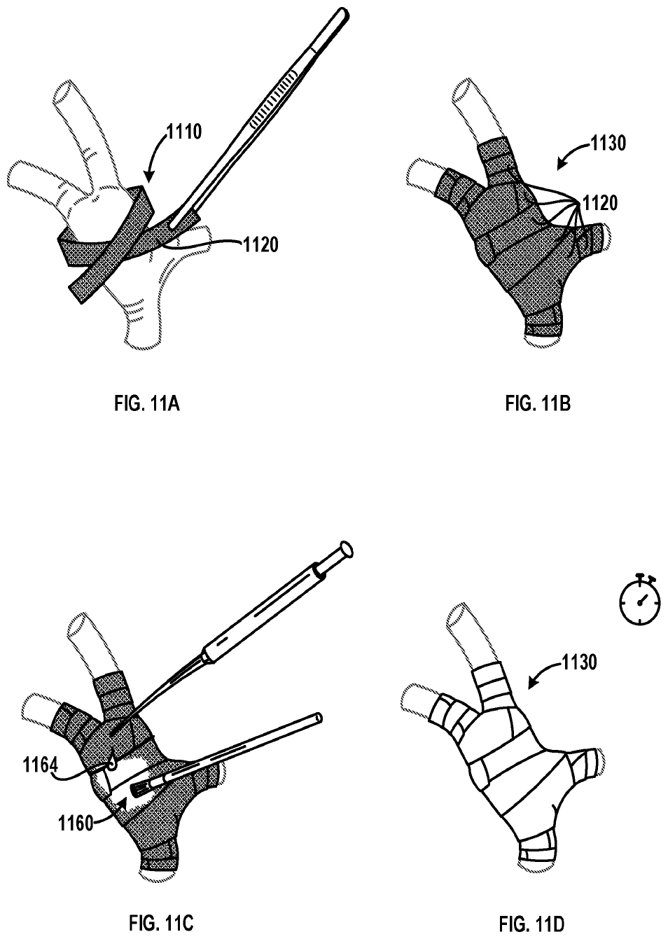

FIGS. 11A-11D show a fourth sequence for vessel reinforcement using the kit outlined herein. In this example, a plurality of sheets 1120 of a framing material 1110 can include the first substance embedded within the framing material 1110. Then as shown in FIG. 11B, the sheets 1120 can then be applied in-situ in an overlapping, piecemeal arrangement around a vessel to form a reinforcing frame 1130. Following formation of the reinforcing frame 1130, as shown in FIG. 11C, a second substance 1164 of the solidifying substance 1160 can be applied to the reinforcing frame 1130 having the first substance until the reinforcing frame 1130 is fully saturated with the combined first substance and the second substance 1164. Once the reinforcing frame 1130 is complete and has been fully saturated with the solidifying substance 1160, as shown in FIG. 11D, then the reinforcing frame 1130 can be allowed to cure until the resulting encasement is fully solidified around the vessel.

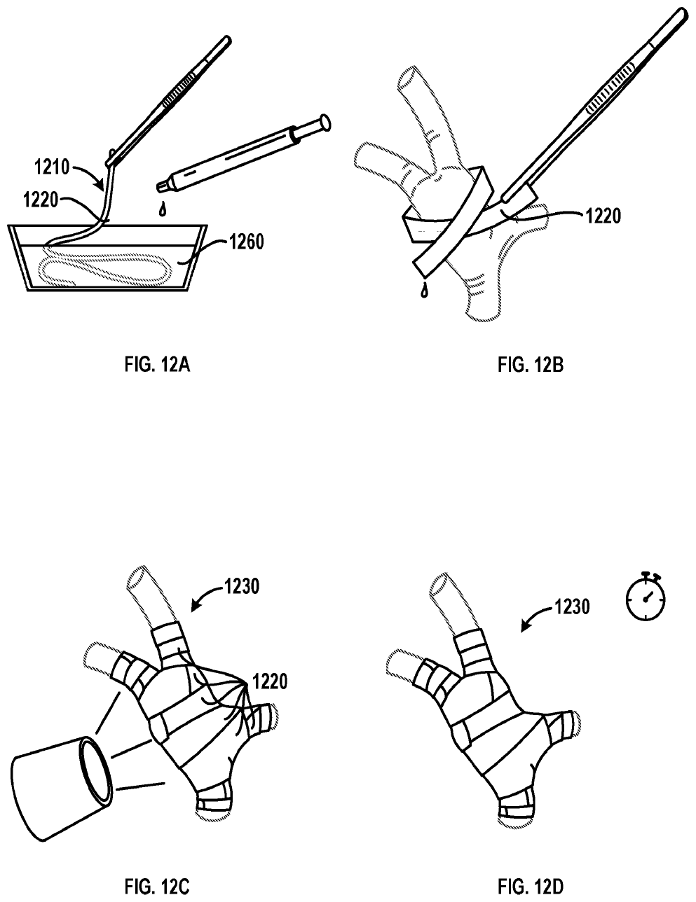

FIGS. 12A-12D show a fifth sequence for vessel reinforcement using the kit outlined herein. As shown in FIG. 12A, a plurality of sheets 1220 of a framing material 1210 can first be soaked in a solidifying substance 1260 (which can be single-part or multi-part as discussed herein) until fully saturated. In some examples, the plurality of sheets 1220 can already be pre-soaked within the solidifying substance 1260. Then as shown in FIG. 12B, the sheets 1220 can then be applied in-situ in an overlapping, piecemeal arrangement around a vessel to form a reinforcing frame 1230. Following formation of the reinforcing frame 1230, as shown in FIG. 12C, a curing step can be applied to the reinforcing frame 1230 to cause the solidifying substance 1260 and the reinforcing frame 1230 to solidify into the encasement. In some examples, the curing step can include application of UV light or another curing element to the solidifying substance 1260 such that the solidifying substance 1260 is caused to transition into the solid state. FIG. 12D shows the resulting encasement having fully solidified around the vessel following the curing step.

### Materials

In one aspect, the solidifying substance and framing material should both be biocompatible. It is understood that components of the solidifying substance and/or framing material may not be biocompatible until combined; in this case, care should be taken to ensure that components of the solidifying substance and/or framing material are sufficiently combined prior to application.

In another aspect, the solidifying substance and framing material should both be radiolucent when combined and when cured to a degree such that the resultant encasement does not interfere with imaging (e.g., X-ray, CT, MRI, Ultrasound, Angiogram, etc.) but may still be at least partially visible under imaging to enable practitioners to assess a condition of the resulting encasement while not obstructing view of the vessel.

Further, the solidifying substance and framing material should adhere to itself. In some examples, the solidifying substance and framing material may not adhere to the vessel, but can instead be wrapped around in such a way that the solidifying substance and framing material contact and form the reinforcing frame around the vessel. When wrapping the framing material around the vessel, it is imperative to ensure that the framing material sufficiently overlaps and “holds” itself together. Hydrophobic materials may be beneficial in ensuring easy cleanup of the surgical space. Further, the solidifying substance and framing material can be removable if necessary; hydrophobic materials may be ideal for this aspect. Care should be taken to avoid damaging the tissues of the vessel or surrounding structures, as such, the solidifying substance and framing material may not permanently adhere to the vessel when solidified.

When combined and when allowed to cure, the solidifying substance and framing material should form an encasement that can withstand pressure of an expanding vessel; e.g., the resulting encasement should be able to withstand high blood pressures (˜180 mmHg).

When combined or applied, it is desired that a resultant reaction does not produce a high thermal output (e.g., a thermal output that cannot be sufficiently and reliably mitigated by surgical techniques such as irrigation) to avoid damaging surrounding tissues.

For the curing process, it is preferred that the solidifying substance and framing material can harden within a reasonable time frame-typically within 2-20 minutes. This range may give practitioners enough time to place and/or adjust the framing material around the vessel while ensuring that the resulting encasement is completely solidified within 20 minutes of placement.

In some embodiments, the solidifying substance and framing material can be further subjected to a curing step following combination and application to the vessel. For instance, the solidifying substance can include a light-curing material that, when exposed to light (e.g., UV light or light within a certain intensity range and/or wavelength), causes the solidifying substance and framing material to solidify and form the encasement. One challenge with this method would be ensuring that the entire mass can be sufficiently exposed to light to allow curing; in some embodiments, the kit may include a fiber-optic light source configured for illuminating small surgical spaces that can accelerate or otherwise facilitate curing when illuminating the solidifying substance and framing material.

In some embodiments, the solidifying substance and framing material can be completely non-biodegradable. In other embodiments, the solidifying substance can be biodegradable after a certain amount of time; in this case, the solidifying substance and/or framing material can be impregnated with a material or drug that facilitates growth of scar tissue along an exterior of the vessel. In some embodiments, the solidifying substance can degrade at a rate that is slow enough to ensure sufficient tissue growth around the exterior of the vessel while the framing material may remain as a bio-scaffold for new tissue.

In a further aspect, the solidifying substance and/or the framing material can include one or more chemicals and/or drugs to promote good surgical outcomes (such as anti-inflammatory and/or antibacterial medications, etc.).

The solidifying substance and framing material should interact well with one another. Specifically, the framing material should be able to sufficiently absorb the solidifying substance. Likewise, the solidifying substance should be able to sufficiently permeate the framing material and extend all the way through the encasement to the vessel and to non-diseased parts of the vessel to ensure full encasement. For instance, if the framing material is hydrophobic, then the solidifying substance should also be hydrophobic. Similarly, if the framing material is hydrophilic, then the solidifying substance should also be hydrophilic.

In some examples, the solidifying substance and/or framing material can have coloring that enables a practitioner to visually confirm whether or not the solidifying substance has permeated the framing material. For instance, the solidifying substance may have a color that, when applied to the framing material, visually shows which areas of the framing material have been saturated in the solidifying substance. In a further aspect, the solidifying substance can undergo a color change when combined (or otherwise activated), when applied to the framing material, and/or when transitioning to the solid state. Similarly, the framing material can undergo a color change when permeated with the solidifying substance and/or when the solidifying substance transitions to the solid state.

A single-part solidifying substance can react with a substance or property of the environment that is not physically bonded to the solidifying substance but is either naturally present in the environment or added to the environment to allow solidifying/polymerization to occur. For example, cyanoacrylate reacts with moisture to polymerize. Light curable materials react with light to solidify.

Solidifying substances that are contemplated herein include but are not limited to: cyanoacrylates (e.g., N-butyl cyanoacrylate for a hydrophobic option, methyl cyanoacrylate for a hydrophilic option), acrylics, hydrogels (e.g., non-biodegradable or slow degradable hydrogels), poly(propylene glycol) diacrylate and pentaerythritol tetrakis(3-mercaptopropionate) (PPODA-QT), silicone-based materials, multi-part epoxies, polyurethanes, epoxy-polyurethane blends, fibrin-based hemostatic adhesives, and/or bio-adhesives. Solidifying substances may include single or multi-part liquids that are operable for curing or otherwise transitioning to a solid state within a predetermined time period, preferably at or less than 20 minutes. For example, a single part solidifying substance reacts with the environment (e.g., water/moisture, light, temperature, etc.) to cure, while the parts of a multi-part solidifying substance react together to cure the solidifying substance. Cure time can be varied by changing the ratios of reagents, temperature, and acidity. It may be possible to add a catalyst in some instances to speed up the reaction. The solidifying substance can cure over the predetermined amount of time after combining individual parts; alternatively, the first substance can remain in a wet (e.g., non-solid) state until exposed to the second substance at which the resultant mixture immediately solidifies.

Framing materials that are contemplated herein include but are not limited to: woven fabrics, nonwoven fabrics, natural fibers (e.g., cotton, wool, etc.), polymers, collagen sheets, cellulose, or bio-based materials. Framing materials may be flexible, conformable, and porous. some embodiments, the framing material may be impregnated with an embedded reactant material that can be activated and hardened upon application of one or more components of the solidifying substance.

In one example, the solidifying substance can include N-butyl cyanoacrylate; the corresponding framing material can include polypropylene.

In another example, the solidifying substance can include PPODA-QT (e.g., including a first substance PPODA for combination with a second substance QT); the corresponding framing material can include polypropylene.

In a further example, the solidifying substance can include 2-octyl cyanoacrylate; the corresponding framing material can include polypropylene or cotton.

### Testing

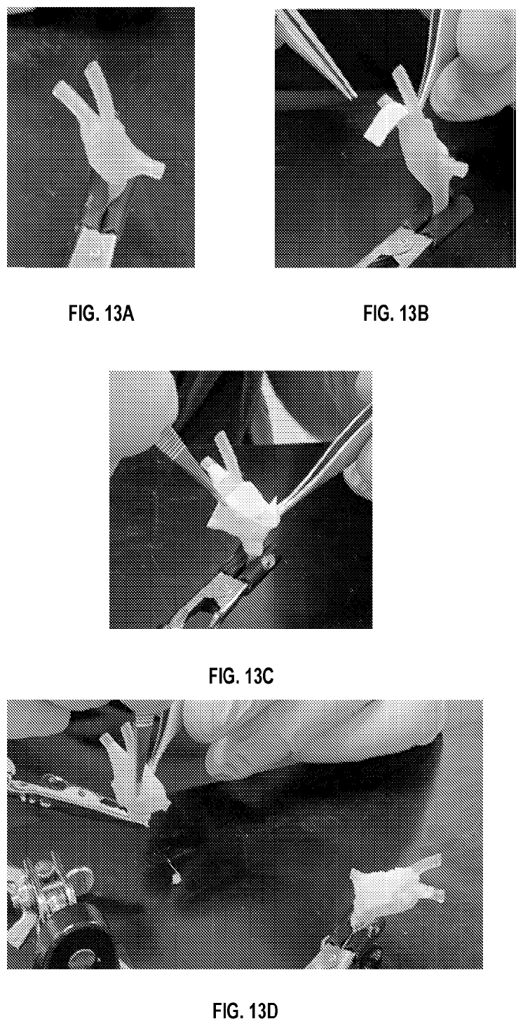

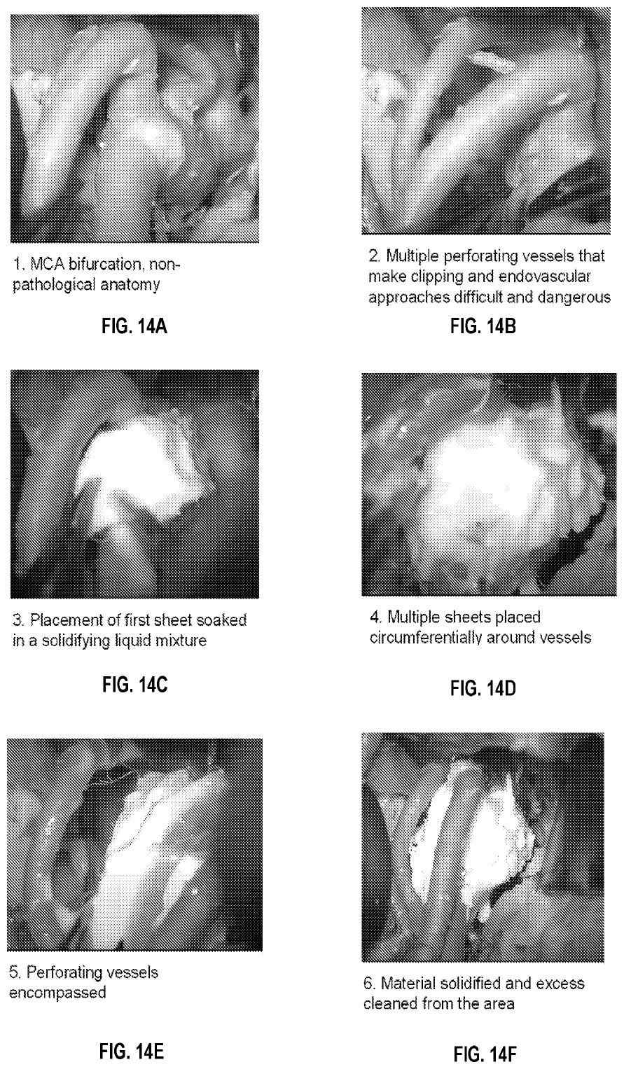

The photographs shown in FIGS. 13A-13D and FIGS. 14A-14F show testing of the sequence shown in FIGS. 8A-8D in which the framing material is soaked in a fully-combined multi-part solidifying compound prior to application.

FIGS. 13A-13D show construction of the reinforcing frame and resulting encasement around a 3-D printed model of a fusiform aneurysm. The framing material selected was spun bound polypropylene cut into rectangular sheets. The solidifying substance was hydrogel (PPODA as a first substance and QT as a second substance, combined to form PPODA-QT). Components of the solidifying substance were first combined to start the solidifying reaction. The sheets were soaked in the solidifying substance, then applied circumferentially to the aneurysm and inflow/outflow vessels. The mixture then solidified, encasing the vessels in a solid encasement. One observation was that the sheets did not conform as well as they could have, likely due to spring-back of the dense framing material. For this reason, it may be advantageous to soften a surface of the framing material to loosen the fibers and make the framing material more pliable. The individual sheets of framing material successfully adhered to one another to form the solid encasement.

FIGS. 14A-14F show construction of the reinforcing frame around a vessel within a cranial space of a cadaver. The framing material selected was spun bound polypropylene cut into rectangular sheets, however the framing material was also texturized or softened with sandpaper to loosen the fibers and make the sheets more pliable. The solidifying substance was hydrogel (PPODA as a first substance and QT as a second substance, combined to form PPODA-QT). Components of the solidifying substance were first combined to start the solidifying reaction. The sheets were soaked in the solidifying substance, then applied circumferentially to the aneurysm and inflow/outflow vessels. The mixture then solidified, encasing the vessels in a solid encasement. One observation was that due to the softened framing material, the framing material conformed more readily to the shape of the vessel. Dispersion of the solidifying substance happened during this test, but was easily re-captured and cleaned. Generally, the reinforcing frame was easy to apply in the presence of water (note that hydrogel and polypropylene are both hydrophobic materials). The individual sheets of framing material successfully adhered to one another to form the solid encasement. Further, the encasement was removable without damage to the surrounding tissues.

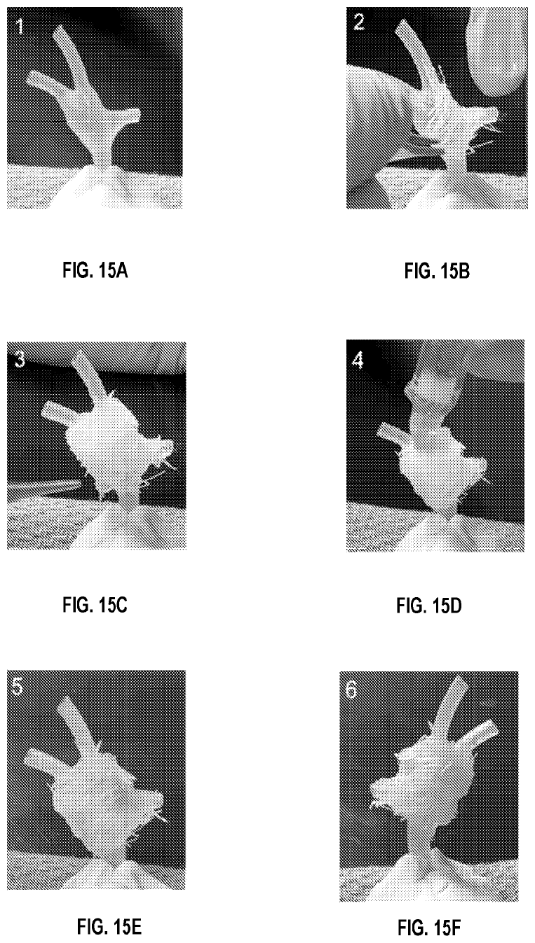

The photographs shown in FIGS. 15A-15F show construction of the reinforcing frame and resulting encasement around a 3-D printed model of a fusiform aneurysm according to the sequence of FIGS. 9A-9D. The framing material selected was loosely woven cotton cut into small sheets. The solidifying substance was cyanoacrylate (a single-part, biocompatible adhesive that cures at room temperature). The framing material was first applied circumferentially to the aneurysm and inflow/outflow vessels to form the reinforcing frame. In one attempt, the framing material was dampened prior to application to make the framing material more receptive to the solidifying substance. The solidifying substance was then applied to the reinforcing frame until the reinforcing frame was fully saturated. The solidifying substance was allowed to cure and solidify, encasing the vessels in a solid encasement. One observation was that the cyanoacrylate was able to permeate through the cotton material relatively well. The cyanoacrylate had a coloring that enabled one to visually confirm areas of the reinforcing frame that have been saturated.

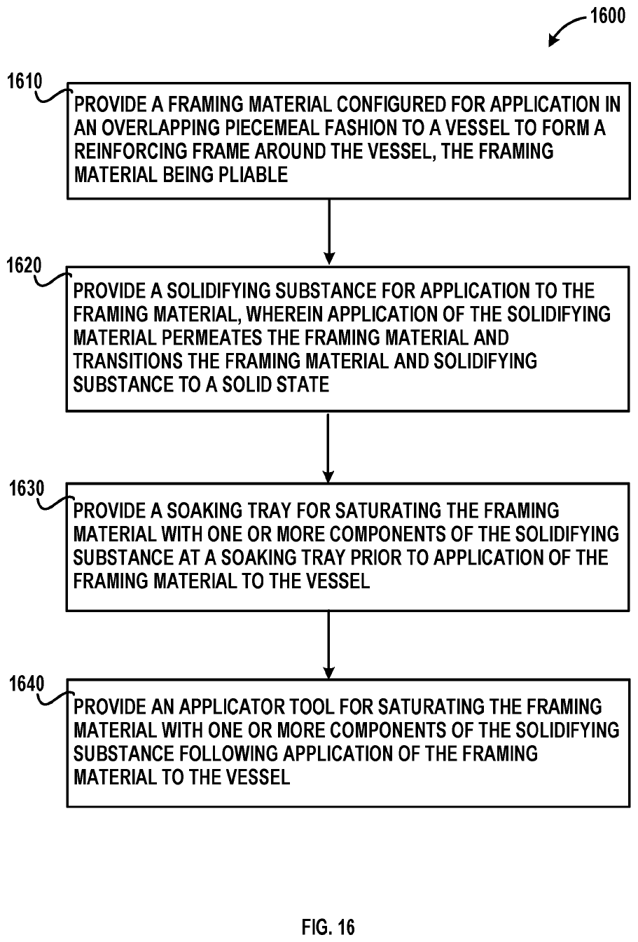

FIG. 16 shows a method 1600 for providing a kit (e.g., kit 100) for vessel reinforcement. Block 1610 of method 1600 includes providing a framing material configured for application in an overlapping piecemeal fashion to a vessel to form a reinforcing frame around the vessel, the framing material being pliable. Block 1620 of method 1600 includes providing a solidifying substance for application to the framing material, where application of the solidifying substance permeates the framing material and transitions the framing material and solidifying substance to a solid state. Block 1630 of method 1600 includes providing a soaking tray for saturating the framing material with one or more components of the solidifying substance at a soaking tray prior to application of the framing material to the vessel. Block 1640 of method 1600 includes providing an applicator tool for saturating the framing material with one or more components of the solidifying substance following application of the framing material to the vessel.

Further, the kit 100 for vessel reinforcement can be applied for treatment of various diseases and/or conditions including but not limited to aneurysms of blood vessels within the body. These can include aortic aneurysms (including abdominal aortic aneurysms and thoracic aortic aneurysms), cerebral aneurysms, common lliac artery aneurysm, femoral and popliteal artery aneurysms.

The kit 100 can be applied for treatment of aneurysms having simple or complex morphologies, such as saccular aneurysms and fusiform aneurysms. The kit 100 can be applied for reinforcing a biological vessel as part of an overall treatment regimen for a condition associated with and/or susceptibility to an aneurysm. While various examples of specific conditions or diseases are provided, it is appreciated that the tools and sequences outlined herein are not limited to these specific listed conditions and diseases, as readily apparent to one of skill in the art.

While there have been shown and described illustrative embodiments of a kit for vessel reinforcement, showing specific sequences, tools, and views, it is to be understood that various other adaptations and modifications may be made within the spirit and scope of the embodiments herein. For example, the various embodiments have been shown and described herein as having specific features, however such features may be interchangeably included (or excluded) from any of the embodiments disclosed herein. It is also appreciated that the various discrete components or component parts of structures described herein can readily be incorporated or integrated into a larger structure. It is also appreciated that while some embodiments include a plurality or pairs of components (e.g., syringes), it is appreciated that a single component can be used with departing from the spirit and scope of this disclosure.

The foregoing description has been directed to specific embodiments. It will be apparent, however, that other variations and modifications may be made to the described embodiments, with the attainment of some or all of their advantages. Accordingly, this description is to be taken only by way of example and not to otherwise limit the scope of the embodiments herein. Therefore, it is the object of the appended claims to cover all such variations and modifications as come within the true spirit and scope of the embodiments herein.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method, comprising:
providing a framing material configured for application to a vessel to form a reinforcing frame around the vessel; and
providing a solidifying substance for application to the framing material, wherein application of the solidifying substance transitions the framing material and solidifying substance to a solid state.

2. The method of claim 1, wherein the solidifying substance includes a first substance that permeates the framing material when applied to the framing material.

3. The method of claim 2, wherein the solidifying substance includes a second substance that permeates the framing material when applied to the framing material, and wherein combination of the first substance with the second substance of the solidifying substance at the framing material causes the framing material and solidifying substance to collectively transition to the solid state within a predetermined amount of time.

4. The method of claim 3, wherein the first substance and the second substance of the solidifying substance are configured for simultaneous application to the framing material.

5. The method of claim 3, wherein the first substance or the second substance of the solidifying substance is configured for application to the framing material prior to application of the framing material to the vessel and wherein the second substance of the solidifying substance is configured for application to the framing material following application of the framing material on the vessel.

6. The method of claim 3, wherein the first substance and the second substance of the solidifying substance are configured for simultaneous application to the framing material prior to wrapping the framing material around the vessel.

7. The method of claim 1, wherein the framing material includes a first substance embedded therein, and wherein combination of a second substance of the solidifying substance with the first substance at the framing material causes the framing material and solidifying substance to collectively transition to the solid state.

8. The method of claim 1, further comprising:
providing a soaking tray for saturating the framing material with one or more components of the solidifying substance at a soaking tray prior to application of the framing material to the vessel.

9. The method of claim 1, further comprising:
providing an applicator tool for saturating the framing material with one or more components of the solidifying substance following application of the framing material to the vessel.

10. The method of claim 1, wherein the framing material includes a plurality of sheets, each sheet being individually configured for wrapping around and/or applying to the vessel.

11. A kit for vessel reinforcement, comprising:
a container, the container being sterile;
a framing material, the framing material being pliable and operable for positioning around a vessel to form a reinforcing frame; and
a solidifying substance that, when applied to the framing material, permeates the framing material and transitions from a non-solid state to a solid state.

12. The kit of claim 11, the framing material having a high permeability with respect to the solidifying substance such that application of a sufficient amount of the solidifying substance to the framing material causes the solidifying substance to permeate the framing material.

13. The kit of claim 11, the solidifying substance having a high absorption with respect to the framing material such that application of a sufficient amount of the solidifying substance to the framing material causes the framing material to absorb the solidifying substance.

14. The kit of claim 11, wherein the solidifying substance includes a first substance and a second substance, wherein combination of the first substance and the second substance causes the solidifying substance to transition from a non-solid state to a solid state.

15. The kit of claim 11, wherein the framing material includes a first substance of the solidifying substance embedded therein that, when combined with a second substance of the solidifying substance, causes the solidifying substance to transition from the non-solid state to the solid state.

16. The kit of claim 11, further comprising:
an applicator configured for application of a first substance and/or a second substance of the solidifying substance to the framing material.

17. The kit of claim 16, wherein the applicator includes a syringe configured for delivery of the solidifying substance to the framing material.

18. The kit of claim 11, wherein the framing material and the solidifying substance are biocompatible.

19. The kit of claim 11, further comprising:
a soaking tray for receipt of one or more components of the solidifying substance and the framing material for saturating the framing material with the one or more components of the solidifying substance.

20. The kit of claim 11, wherein the framing material includes a pliable fabric material.

21. A method of reinforcing a biological vessel, comprising:
applying a framing material to a vessel to form a reinforcing frame around the vessel; and
applying a solidifying substance to the framing material, wherein application of the solidifying substance permeates the framing material and transitions the framing material and solidifying substance to a solid state.

22. The method of claim 21, wherein the vessel has an aneurysm having a saccular morphology.

23. The method of claim 21, wherein the vessel has an aneurysm having a fusiform morphology.

24. The method of claim 21, wherein the vessel is an aortic vessel having an aortic aneurysm.

25. The method of claim 21, wherein the vessel is a cerebral vessel having a cerebral aneurysm.

26. The method of claim 21, wherein the vessel is a common Iliac artery having an Iliac aneurysm.

27. The method of claim 21, wherein the vessel is a femoral artery having a femoral aneurysm.

28. The method of claim 21, wherein the vessel is a popliteal artery having a popliteal aneurysm.

29. The method of claim 21, wherein reinforcing the biological vessel is part of an overall treatment regimen for a condition associated with and/or susceptibility to an aneurysm.

30. The method of claim 1, where the framing material is configured for application to the vessel an overlapping piecemeal fashion.

31. The method of claim 1, the framing material being pliable.

32. The kit of claim 11, the container configured for secure storage and transport of the solidifying substance and the framing material.

33. The method of claim 21, further comprising:
applying the framing material to the vessel in an overlapping piecemeal fashion to form the reinforcing frame around the vessel.

34. The method of claim 21, the framing material being pliable.
